SEQUENCE LISTING (5 sequences)

SEQ ID NO: 1 - protein, 22 aa, Homo sapiens
SPAVEQQLLVSGPGKKPEDSRV

SEQ ID NO: 2 - DNA, 21 bp, Artificial Sequence
  Synthetic construct
tccagcagtcatgggacccca

SEQ ID NO: 3 - DNA, 20 bp, Artificial Sequence
  Synthetic Construct
cccaagacagctcacactcg

SEQ ID NO: 4 - DNA, 20 bp, Artificial Sequence
  Synthetic Construct
actccagcagtcatgggacc

SEQ ID NO: 5 - DNA, 22 bp, Artificial Sequence
  Synthetic Construct
acgagtaggattctcctcactt

(19) United States
(12) Patent Application Publication
(10) Pub. No.: US 2008/0038728 A1
(43) Pub. Date: Feb. 14, 2008

(54) Quadruple Transformed Cell Line Useful for the Identification of Transport Inhibitors

(76) Inventors: Kathrin De Jong, Bad Schwalbach (DE); Jorg Konig, Louuerstadt (DE); Dietrich Keppler, Dossenheim (DE)

Correspondence Address: MOORE & VAN ALLEN PLLC, P.O. BOX 13706, Research Triangle Park, NC, 27709, US

(21) Appl. No.: 11/576,819

(22) PCT Filed: Oct. 7, 2005
(86) PCT No.: PCT/EP05/10842

§ 371 Date: Sep. 18, 2007

(30) Foreign Application Priority Data
Oct. 7, 2004 (EP) 04023917.0

(51) Int. Cl. C12Q 1/68; C12N 15/63; C12N 5/06; C12N 5/08

(52) U.S. Cl. 435/6; 435/325; 435/350; 435/366; 435/455

(57) ABSTRACT

Described is a cell line containing (a) three DNA sequences encoding different uptake transporters for organic anions, preferably OATP1B1, OATP1B3 and OATP2B1, operatively linked with a promoter and (b) a DNA sequence encoding an export pump for organic anions or anionic conjugates, preferably the multidrug resistance protein 2 (MRP2), operatively linked with a promoter. Moreover, various uses of said cell line are described, preferably for the identification of transport inhibitors, e.g. drug candidates.

The present invention relates to a cell line containing (a) three DNA sequences encoding different uptake transporters for organic anions, preferably OATP1B1 (=OATP2), OATP1B3 (=OATP8) and OATP2B1 (=OATP-B), operatively linked with a promoter and (b) a DNA sequence encoding an export pump for organic anions or anionic conjugates, preferably the multidrug resistance protein 2 (MRP2, ABCC2), operatively linked with a promoter. The present invention also relates to various uses of said cell line, preferably for the identification of transport inhibitors, e.g. drug candidates.

Vectorial transport is an important function of all polarized cells contributing to detoxification and to the prevention of entry of toxins into organs. This is exemplified by kidney proximal tubule epithelia, by cells of the blood-brain barrier, by intestinal epithelia, and, last but not least, by hepatocytes. One of the major hepatocellular functions is the removal of endogenous and exogenous substances from the blood circulation and their secretion into the bile. Two transport processes play a decisive role in this vectorial transport by hepatocytes: the sinusoidal (basolateral) uptake from blood and the canalicular (apical) secretion into bile. In human hepatocytes, the sodium-independent uptake of amphiphilic organic anions is, mediated by at least three transport proteins, namely by the human organic anion transporters OATP1B1 (also known as OATP2, OATP-C or LST1, symbol SLC01B1 or SLC21A6) (Abe et al., J. Biol. Chem. 274 (1999), 17159-63; Hsiang et al., J. Biol. Chem. 274 (1999), 37161-8; König et al., Am. J. Physiol. Gastrointest. Liver Physiol. 278 (2000), G156-64; Cui et al., J. Biol. Chem. 276 (2001), 9626-30), human OATP1B3 (OATP8, SLCO1B3 or SLC21A8) (König et al., J. Biol. Chem. 275 (2000), 23161-8; Cui et al., Mol. Pharmacol. 60 (2001), 934-43)), and human OATP2B1 (OATP-B, SLCO2B1 or SLC21A9) (Kullak-Ublick et al., Semin. Liver Dis. 20 (2000), 273-93). All three transporters belong to the subgroup SLCO (or 21A) of the solute carrier (SLC) superfamily. Whereas OATP2B1 is expressed also in a number of other tissues, OATP1B1 and OATP1B3 are expressed exclusively in human hepatocytes. The substrate spectrum of OATPs includes bile salts, conjugates of steroid hormones, thyroid hormones, and many other amphiphilic organic anions (Abe et al. (1999); Cui et al. (2001); Kullak-Ublick et al. (2000), Gastroenterology 120 (2001), 525-33). Unlike these basolateral uptake transporters which are thought to be of exchanger type, the apical export transporters identified in human hepatocytes so far are members of the ATP-binding cassette (ABC) superfamily. The export of organic anions is predominantly mediated by the bile salt export pump BSEP (ABCB11) belonging to the MDR (ABCB) subgroup of the ABC superfamily and by the multidrug resistance protein 2 (MRP2, ABCC2) belonging to the MRP (ABCC) subgroup of the ABC superfamily. While the major substrates of BSEP are bile salts like cholyl taurine and cholate, the organic anions transported by MRP2 are mainly conjugates of lipophilic substances with glutathione, glucuronate, or sulphate.

The transhepatic transport of amphiphilic organic anions has been frequently studied by use of model compounds like sulfobromophthalein (BSP) and indocyanine green (ICG) (Scharschmidt et al., J. Clin. Invest. 56 (1975), 1280-1292). Functional characterization of the three human OATPs identified in the hepatocyte basolateral membrane demonstrated that all three are able to mediate the uptake of BSP, with the highest affinity for OATP1B1 (Kₘ=140 nM) and the lowest affinity (Kₘ=3.4 μM) for OATP1B3. In the hepatocyte BSP is predominantly conjugated with glutathione to yield the BSP glutathione S-conjugate (BSP-SG). Studies with transport-deficient mutant rats lacking functional MRP2 in the apical membrane suggested that this export pump mediates the secretion of BSP-SG into bile.

When developing or designing new pharmaceuticals one of the critical questions is to whether the candidate compounds have undesired side effects like interference with the transport of substances which are produced or occur naturally in the body, e.g. interference with the hepatic or renal transport of organic anions, as exemplified by the interference of rifampicin, rifamycin SV, or CDNB with the transcellular transport of BSP. For cost saving, it is desirable to detect such side effects of drug candidates at an early stage of development. So far, for the study of potential side effects of drug candidates animals or cell cultures were used. However these approaches exhibit a variety of disadvantages, e.g. are time and cost consuming, do not allow the high throughput screening of drug candidates etc. Moreover, previously double-transfectants based on MDCK cells were constructed for screening candidate drugs (Cui et al., Mol. Pharmacol. 60 (2001), 934-43: OATP1B3+MRP2); Sasaki et al. (2002), J. Biol. Chem. 277, 6497-6503: OATP1B1+MRP2). However, the reliable identification of substrates that are transported only by one of the OATPs requires the use of three different cell lines (double-transfectants). Unfortunately, such cell lines are not available and, of course, screening procedures requiring the use of three different cell lines are cost-intensive and time-consuming.

Hepatobiliary elimination of many organic anions is initiated by OATP1B1 (OATP2, LST-1, OATP-C), OATP1B3 (OATP8), and OATP2B1 (OATP-B), which are the predominant uptake transporters of human hepatocytes. Subsequently, the unidirectional efflux pump ABCC2 (multidrug resistance protein 2) mediates the transport of organic anions, including glutathione conjugates and glucuronosides, into bile.

Therefore, it is the object of the present invention to provide a means for the efficient analysis of the interference of a drug candidate with transport of substances which are produced or occur naturally in the body, particularly interference with the hepatic or renal transport of organic anions which overcomes the disadvantages of the systems presently used.

According to the invention this is achieved by the subject matters defined in the claims.

A quadruple transfected cell line with defined human uptake and export transporters was established, i.e. a quadruple-transfected MDCKII cell line permanently expressing three recombinant uptake transporters for organic anions in the basolateral membrane and an ATP-dependent export pump for anionic conjugates in the apical membrane. Basolateral uptake was mediated by the human organic anion transporters OATP1B1, OATP1B3 and OATP2B1 that are the predominantly expressed transporters for organic anions in human hepatocytes and subsequent apical export by the multidrug resistance protein 2 (MRP2; ABCC2). Under physiological conditions, these transport proteins are strongly expressed in hepatocytes and contribute to the hepatobiliary elimination of organic anions. Expression and localization of OATP1B1, OATP1B3, OATP2B1 and MRP2 in MDCK cells growing on Transwell membrane inserts was demonstrated by immunoblotting and confocal laser scanning microscopy.

To conclude, the cell lines of the present invention for the first time provide a system allowing to reliably determine in a single screening experiment which drug candidates, in particular organic anions, are taken up by hepatocytes and transported into the gallbladder. Moreover, the cell lines of the present invention for the first times allows to determine interferences of different drugs not only on the level of uptake transport but also on the level of the MRP2 mediated excretion into the gallbladder. A further advantage of the cell system of the present invention vis-à-vis the presently used cells is that it in case of interference of the drug candidate with one of the transport proteins the remaining OTPs are capable of compensating the transport deficiency. Results obtained by this approach yield important information for the assessment of drug interferences and, thus, allow to make predictions for potential side effects. Finally, by use of the cell lines of the present invention an improved characterization of the substrate specificity of the apical ATP-dependent transporter MRP2 can be made which is due to the different substrate specificities of the three OATP transporters. The cell lines of the present invention are particularly useful for high throughput screening methods.

Accordingly, the present invention provides a cell line, preferably a stable cell line containing (a) three DNA sequences encoding different uptake transporters for organic anions operatively linked with a promoter and (b) a DNA sequence encoding an export pump for organic anions or anionic conjugates operatively linked with a promoter. Preferably, the uptake transporter for organic anions is expressed in the basolateral cell membrane and the export pump for organic anions or anionic conjugates is expressed in the apical cell membrane.

In certain cases, e.g., for investigating the effects of the intracellular drug metabolism on transport it might be desirable to transfect the cells of the invention with one or more additional DNA sequences encoding polyppetides the nature of which may vary according to the drug to be studied, e.g., a further transporter or metabolizing enzyme.

Preferably, for transfecting the cells the DNA sequences are present in a vector or expression vector. A person skilled in the art is familiar with examples thereof. The DNA sequences can also be contained in a recombinant virus containing appropriate expression cassettes. Suitable viruses that may be used in the present invention include baculovirus, vaccinia, sindbis virus, SV40, Sendai virus, adenovirus, an AAV virus or a parvovirus, such as MVM or H-1. The vector may also be a retrovirus, such as MoMULV, MoMuLV, HaMuSV, MuMTV, RSV or GaLV. For expression in mammals, a preferred suitable promoter is the human cytomegalovirus “immediate early promoter” (pCMV).

For generating the above described DNA sequences and for constructing expression vectors which contain said DNA sequences, it is possible to use general methods known in the art. These methods include e.g. in vitro recombination techniques, synthetic methods and in vivo recombination methods as described in Sambrook et al., Molecular Cloning, A Laboratory Manual, 2^{nd} edition (1989) Cold Spring Harbor Laboratory Press, Cold Spring Harbor, N.Y., for example. Methods of transfecting the cells, of phenotypically selecting transfectants and of expressing the DNA according to the invention by using the above described vectors are also known in the art.

Preferably, the cell line is a human cell line, e.g. HEK293. Particularly preferred are polarized cells, e.g. hepatocytes, kidney cells, e.g. MDCKII or HepG2.

In a further preferred embodiment of the cell line of the present invention the uptake transporters for organic anions are members of the subgroup SLCO (SLC21A) of the solute carrier (SLC) superfamily. Particularly preferred are OATP1B1, OATP1B3 and OATP2B1.

In a more preferred embodiment of the cell line of the present invention the export pump for organic anions or anionic conjugates is a member of the MDR (ABCB) subgroup or the MRP (ABCC) subgroup of the ABC superfamily. Particularly preferred are the bile salt export pump BSEP (ABCB11) or the multidrug resistance protein 2 (MRP2).

In an even more preferred of the cell line of the present invention the DNA sequence encoding an uptake transporter for organic anions and/or the DNA sequence encoding an export pump for organic anions or anionic conjugates are operatively linked with a promoter allowing high expression.

Finally, the present invention relates to various uses of the quadruple-transfected cell line. A preferred use is the identification of a transport substrate or a transport inhibitor, particularly a drug candidate. Suitable assay formats are known to the person skilled and, e.g., described in the examples, below. A preferred assay format is high throughput screening.

### ADVANTAGES OF THE PRESENT INVENTION

In this invention, a MDCKII cell line stably expressing recombinant OATP1B1, OATP1B3, and OATP2B1 in the basolateral membrane and ABCC2 in the apical membrane has been created. Double-transfected MDCKII cells stably expressing ABCC2 together with OATP1B1, or OATP1B3, or OATP2B1 served as controls. The quadruple-transfected cells exhibited high rates of vectorial transport of organic anions including bromosulfophthalein, cholecystokinin peptide (CCK-8), and estrone 3-sulfate. The quadruple-transfected cells enabled the identification of substrates for uptake or vectorial transport that may be missed in studies with a double-transfected cell line, as exemplified by CCK-8 which is a substrate for OATP1B3, but not for OATP1B1 or OATP2B1. The broad substrate spectrum covered by the 3 hepatocellular OATP transporters enables representative analyses of the uptake of many organic anions into human hepatocytes. The broad spectrum of organic anions transported vectorially by the quadruple-transfected cells also provides valuable information on the substrate selectivity of ABCC2, without the need for studies in inside-out membrane vesicles containing the ABCC2 protein. The quadruple-transfected MDCKII-ABCC2/OATP1B1/1B3/2B1 cells may thus be useful for the identification of substrates and inhibitors, including drug candidates, undergoing uptake and secretion by human hepatocytes, under conditions that may be better defined than in primary cultures of human hepatocytes.

In the present invention a preferably quadruple-transfected MDCKII cell line stably expressing the uptake transporters OATP1B1, OATP1B3, and OATP2B1, together with the efflux pump ABCC2 has been established. As shown by immunoblot analysis, all 4 transport proteins were expressed at high levels in the ABCC2/OATP1B1/1B3/2B1 quadruple-transfected MDCKII cells (FIG. 5). Confocal laser scanning immunofluorescence microscopy indicated the basolateral localization of OATP1B1 and OATP1B3 and the apical localization of ABCC2 in the MDCKII transfectants, thus confirming previous localization studies (Evers et al., 1998, König et al., 2000a, Cui et al., 2001, Sasaki et al., 2002). MDCKII cells stably expressing OATP2B1 are novel, and this protein was localized to the basolateral membrane both in the ABCC2/OATP2B1 double-transfectants and in the ABCC2/OATP1B1/1B3/2B1 quadruple-transfected cells.

By using BSP as a common substrate it has been demonstrated that all recombinantly expressed transporters in the quadruple-transfected cells are functionally active in transcellular BSP transport (FIG. 8). The rates of transcellular BSP transport by the double-transfected MDCKII cell lines, ABCC2/OATP1B1, ABCC2/OATP1B3, and ABCC2/OATP2B1, were relatively low at 100 nM BSP, however, an increased intracellular accumulation of BSP was measurable even at this low substrate concentration (FIG. 8), probably as a result of the lower affinity of BSP for ABCC2 (Kₘ 12 μM; Cui et al., 2001) as compared to the OATPs (Km values<5 μM; Table 1). In comparison to the double-transfectants, the ABCC2/OATP1B1/1B3/2B1 quadruple-transfectants showed a significantly increased intracellular accumulation and transcellular transport even at 100 nM BSP. The transcellular transport was also highest in the quadruple-transfected cells at 5 μM BSP when compared to any of the double-transfected MDCKII cells (FIG. 8). The export of BSP via ABCC2 may depend on a threshold concentration of intracellularly accumulated BSP after uptake via the OATP proteins. If 3 OATP proteins act together in one cell to accumulate BSP intracellularly, as it is the case in the quadruple-transfectants, this threshold concentration of intracellular BSP is achieved earlier. Moreover, transcellular transport of CCK-8 was examined in the MDCKII transfectants. Both ABCC2/OATP1B3 double-transfected MDCKII cells and ABCC2/OATP1B1/1B3/2B1 quadruple-transfectants transported CCK-8 (FIG. 9), but not the ABCC2/OATP1B1 and ABCC2/OATP2B1 double-transfectants. These data confirm that CCK-8 is a selective substrate for OATP1B3 with a Kₘ value of 11.1 μM (Ismair et al., 2001) and a substrate for ABCC2 with a Kₘ value of 8.1 μM (Letschert et al., 2005). These results also demonstrate that the transcellular transport of a compound that is a selective substrate for only one of the OATP proteins will be detected by the ABCC2/OATP1B1/1B3/2B1 quadruple-transfectants, whereas only one of the 3 double-transfectants would show a significant transcellular transport. Since the affinities of OATP1B3 and ABCC2 for CCK-8 are in a similar range, the cellular uptake of CCK-8 proceeded equally well as the export via ABCC2 (Letschert et al., 2005). It has been observed that estrone 3-sulfate, an established substrate for OATP1B1, OATP1B3, and OATP2B1 (Kullak-Ublick et al., 2001), is transported transcellularly by the double-transfectants and by the quadruple-transfectants (FIG. 9). The affinity to estrone 3-sulfate is higher for OATP1B1 than for OATP2B1 (Tamai et al., 2000). This is in line with the observation that the uptake rate of 1 μM estrone 3-sulfate in the ABCC2/OATP1B1 double-transfectants is higher than in the ABCC2/OATP1B3 or the ABCC2/OATP2B1 double-transfectants (FIG. 9). Estrone 3-sulfate is also a substrate for ABCC2, as evidenced by its vectorial transport (FIG. 9) and by recent data on transcellular estrone 3-sulfate transport in LLC-PK1 cells double-transfected with OATP1B1 and ABCC2 (Spears et al., 2005). Moreover, it has been demonstrated directly by membrane vesicle studies that estrone 3-sulfate is a substrate for ABCC2 (FIG. 10).

In the present invention, fluvastatin was identified as a substrate for OATP1B1, OATP1B3, and OATP2B1. The highest affinity was determined with the ABCC2/OATP2B1 double-transfectants (Kₘ 0.7 μM), suggesting that OATP2B1 plays an important role in statin uptake into hepatocytes. The ABCC2/OATP1B1/1B3/2B1 quadruple-transfectants transported fluvastatin with a Kₘ value of 2.3 μM (Table 1). Also, the most efficient uptake and intracellular accumulation were observed in the quadruple transfectants. A vectorial transport of fluvastatin into the apical compartment could not be detected, suggesting that fluvastatin is not a substrate for ABCC2. This conclusion was supported by studies using inside-out membrane vesicles from MDCKII-ABCC2 single-transfected cells (not shown). Most of the statins acting as inhibitors of HMG-CoA reductase, including fluvastatin, act in hepatocytes and are metabolized there in part, followed by excretion into bile (Tse et al., 1995). Since it has not been observed a significant transcellular transport of fluvastatin itself in the ABCC2/OATP1B1/1B3/2B1 quadruple-transfectants, either one of its metabolites may be transported by ABCC2 or other ATP-dependent efflux pumps in the apical membrane may be responsible for the in vivo efflux of fluvastatin into bile.

Kinetic analyses of the 5-min uptake transport of BSP by ABCC2/OATP1B1, ABCC2/OATP1B3, and of ABCC2/OATP2B1 double-transfectants indicated Kₘ values for BSP of 2.4 μM, 2.2 μM, and 3.4 μM, respectively (Table 1). This confirms that these 3 OATP proteins have a relatively high affinity for BSP. These Kₘ values determined in the double-transfectants were somewhat higher than the values reported earlier for OATP1B1-, OATP1B3-, and OATP2B1-mediated transport of BSP in cRNA-injected Xenopus laevis oocytes (0.3 μM, 0.4 μM, and 0.7 μM) (Kullak-Ublick et al., 2001). In the ABCC2/OATP1B1/1B3/2B1 quadruple-transfectants BSP was transported with a Kₘ value of 4.8 μM, but the maximal velocity was elevated to 197.8 pmol×mg protein×min⁻¹ which is markedly above the maximal velocity of the double-transfectants (Table 1).

The novel quadruple-transfected MDCKII cells are thus useful in studies on the transport properties of ABCC2, OATP1B1, OATP1B3, and OATP2B1. Common substrates of the 3 uptake transporters, like BSP and fluvastatin, are efficiently taken up into the quadruple-transfectants. Specific substrates for one of the uptake transporters, as demonstrated for CCK-8 and OATP1B3, are easily detected by the ABCC2/OATP1B1/1B3/2B1 quadruple-transfected cells. These cells may be useful in the screening of drugs and drug candidates with respect to their substrate and inhibitor properties in hepatocellular uptake and ABCC2-mediated efflux into bile.

### BRIEF DESCRIPTION OF THE DRAWINGS

FIG. 1: Scheme of the Quadruple Transfectant

After proliferation on a Transwell membrane the cells constitute a single layer. The cells are intercellularly connected by particular “tight-junctions”. Thus, the media of the lower and upper chamber do not have any direct contact and are separated by the cell layer. The polarity of the cells of the quadruple transfectants is schematically shown with the basolateral expression of the transporters OATP1B1, OATP1B3 and OATP2B1 and the apical expression of MRP2.

FIG. 2: Immunolocalization of Recombinantly Expressed transporters MRP2, OATP1B1, OATP1B3 and OATP2B1 in MDCKII cells

See Example 2, for details.

FIG. 3: Transcellular Transport of the Substrate [³H]BSP

See Example 3, for details.

FIG. 4: Scheme of MDCKII Cells Stably Expressing Recombinant ABCC2 and OATP Proteins.

ABCC2 single-transfectant (A), double-transfectants expressing ABCC2 together with OATP1B1 or OATP1B3 or OATP2B1 (B), and quadruple-transfectant expressing ABCC2 together with OATP1B1, OATP1B3, and OATP2B1 (C).

FIG. 5. Immunoblot of ABCC2, OATP1B1, OATP1B3, and OATP2B1 proteins in stably transfected MDCKII cells

Crude membrane fractions from MDCKII cells transfected with the control vector, with ABCC2, with ABCC2 and OATP1B1, with ABCC2 and OATP1B3, with ABCC2 and OATP2B1, and with ABCC2 together with OATP1B1, OATP1B3, and OATP2B1 cDNA (quadruple-transfectant) were separated by SDS-PAGE. OATP1B1 was detected by the antiserum ESL (König et al. 2000a) (A), OATP1B3 was detected by the antiserum SKT (König et al., 2000b) (B), OATP2B1 by the antiserum SPA (C), and ABCC2 by the antiserum EAG5 (Schaub et al. 1999) (D) (see Example 1).

FIG. 6. Immunolocalization of ABCC2 and OATP Proteins

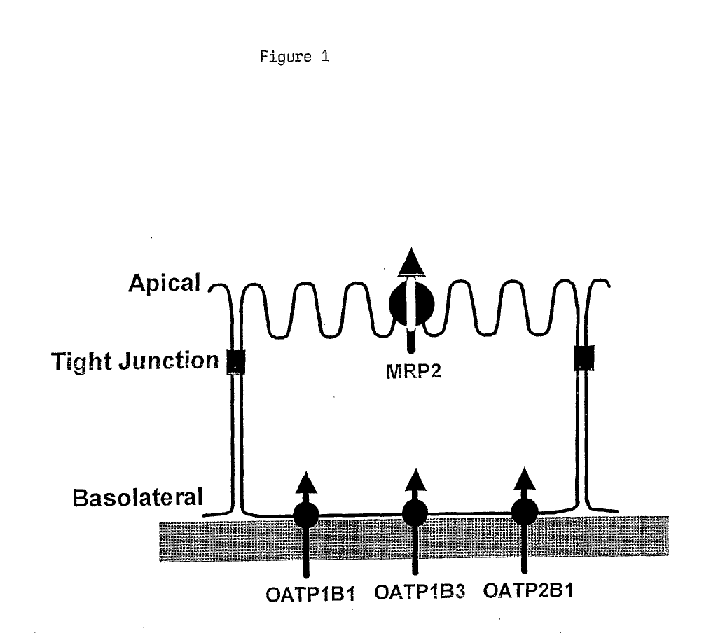

Immunolocalization of ABCC2 and OATP proteins in human liver (A-C) and in transfected MDCKII cells (D-O) by confocal laser scanning immunofluorescence microscopy. Human liver cryosections as well as MDCKII transfectants grown on cell culture inserts were fixed with 10% and 2% formaldehyde solution, respectively. ABCC2 (red fluorescence) was detected with the monoclonal antibody M₂III-6. The OATP proteins are shown by the green fluorescence. OATP1B1 (A, D, G, J, M) was detected by the antiserum ESL (König et al., 2000a). OATP1B3 (B, E, H, K, N) was detected by the antiserum SKT (König et al., 2000b). OATP2B1 (C, F, I, L, O) was detected by the antiserum SPA (see Materials and Methods). D, E, F, J, K, and L are en face images at the top of the cell monolayers. G, H, I, M, N, and O are vertical sections through the cell monolayers at positions indicated by the broken white lines. In human liver, ABCC2 is localized to the bile canaliculi of hepatocytes (A-C), whereas OATP1B1, OATP1B3, and OATP2B1 are localized to the basolateral membrane of hepatocytes. Analogously, in the MDCKII transfectants ABCC2 is localized apically and the OATP proteins are localized basolaterally.

FIG. 7: Vectorial Transport of [³H]BSP

The vector-transfected MDCKII control cells and all transporter-transfected cells listed were grown on cell culture inserts (see Example 1). [³H]BSP (5 μM) was either added to the apical compartment (A) or to the basolateral compartment (B). After 15 min, the radioactivity in the opposite compartment was measured. Data represent means ±SD determined from 3 experiments each performed in triplicate.

FIG. 8: Transport of [³H]BSP.

The vector-transfected MDCKII control cells and the transporter-transfected cells listed were grown as described in Materials and Methods, and incubated with 100 nM [³H]BSP (A, B) or 5 μM [³H]BSP (C, D) in the basolateral chamber. After incubation for 30 min, radioactivity determined in the apical chamber was used to calculate the transcellular transport (A, C). Intracellular substrate accumulation was calculated after lyzing the cells (B, D). Data represent means ±SD determined from 3 experiments each performed in triplicate.

FIG. 9: Transport of [³H]CCK-8, [³H]estrone 3-sulfate (E3S) and [³H]fluvastatin (Fluva).

Cells were grown as described in Materials and Methods, and incubated with 1 nM [³H]CCK-8 (A, B), 1 μM [³H]E3S (C, D) or 0.5 μM [³H]Fluva (E, F). Transcellular transport (A, C, E) and intracellular substrate accumulation (B, D, F) were calculated as means ±SD determined from 3-7 experiments each performed in triplicate.

FIG. 10: Transport of [³H]estrone 3-sulfate (E3S) by ABCC2.

ATP-dependent transport into inside-out membrane vesicles prepared from MDCKII-ABCC2 single-transfectants (ABCC2, closed circles) or vector-transfected MDCKII cells (Control, open circles) was measured for 1, 3, and 5 min as described in Example 1. Data are means ±SD determined from 3 measurements.

The present invention is explained by the following examples.

### EXAMPLE 1

### Materials and Methods

(A) Chemicals

[³H]Bromosulfophthalein ([³H]BSP) (0.6 TBq/mmol) and [³H]fluvastatin (1.55 Ci/mmol) were obtained from Hartmann Analytic (Braunschweig, Germany) by custom synthesis. [³H]Estrone 3-sulfate (2.12 TBq/mmol) and [³H]cholecystokinin-8 sulfate ([³H]CCK-8) (3.52 TBq/mmol) were purchased from Amersham Biosciences (Amersham, UK). Unlabeled fluvastatin was purchased from Sequoia Research Products (Oxford, U.K.). Unlabeled BSP and estrone 3-sulfate were obtained from Sigma (Taufkirchen, Germany). G418 (geneticin) disulfate, hygromycin, zeocin, and blasticidin were from Invitrogen (Groningen, Netherlands). Additional non-radioactive chemicals of analytical purity were obtained from Sigma.

(B) Antibodies.

For detection of ABCC2 the antiserum EAG5 (Büchler et al., 1996; Keppler and Kartenbeck, 1996) and the mouse monoclonal antibody M₂III-6 (Alexis Biochemicals, San Diego, Calif.) were used. OATP1B1 was detected using the antiserum ESL (König et al., 2000a). OATP1B3 was detected by the antiserum SKT (König et al., 2000b). For detection of OATP2B1, the antiserum SPA was raised in rabbits using a peptide corresponding to the carboxyl-terminal amino acids 688-709 of the human OATP2B1 sequence (SPAVEQQLLVSGPGKKPEDSRV; NCBI accession number NP_{—}009187) coupled via an additional N-terminal tyrosine to keyhole limpet hemocyanin (Peptide Specialty Laboratories, Heidelberg, Germany). Horseradish peroxidase-conjugated goat anti-rabbit antibodies were from Bio-Rad (München, Germany). Cy3-conjugated goat anti-mouse IgG was obtained from Jackson Laboratories (West Grove, Pa.) and Alexa Fluor 488-conjugated goat anti-rabbit IgG was from Molecular Probes (Eugene, Oreg.).

(C) Construction of Recombinant Vectors

The cDNA encoding ABCC2 (GenBank/EMBL accession number X96395), originally cloned by Büchler et al. (1996) and Cui et al. (1999) was subcloned into the expression vector pcDNA3.1(+) (Invitrogen). cDNA encoding OATP1B1 (EMBL/GenBank accession number NM_{—}006446) was cloned as described in König et al., Am. J. Physiol. Gastrointest. Liver Physiol. 278 (2000), G156-64, and subcloned into the expression vector pcDNA3.1/Hygro(−). cDNA encoding OATP1B3 (EMBL/GenBank accession number NM_{—}019844) was cloned as described in König et al., J. Biol. Chem. 275 (2000), 23161-8, and subcloned into the expression vector pcDNA3.1/Zeo(+)(Invitrogen). cDNA encoding MRP2 (EMBL/GenBank accession number X96395) was cloned as described in Cui et al., Mol. Pharmacol. 55 (1999), 929-37, and subcloned into the expression vector pcDNA3.1(+) (Invitrogen).

The cDNA encoding OATP2B1 was cloned from human adult brain by PCR using the following OATP2B1-specific primers (forward: 5′-TCCAGCAGTCATGGGACCCCA-3′; reverse: 5′-CCCAAGACAGCTCACACTCG-3′) based on the original sequence published by Tamai et al. (2000) (GenBank/EMBL accession number NM_{—}007256) and cloned into the vector pCR2.1-TOPO (Invitrogen). The cDNA sequence was 100% identical with the reference sequence (GenBank/EMBL accession number NM_{—}007256) and was subcloned into the expression vectors pcDNA3.l/Hygro(−) and pcDNA6/V5-HisB (Invitrogen). Furthermore, cDNA encoding OATP2B1 (EMBL/GenBank accession numbert NM_{—}007256) was amplified using the primers pOATP2B1.for (5′-ACTCCAGCAGTCATGGGACC-3′) and pOATP2B1.rev (5′-ACGAGTAGGATTCTCCTCACTT-3′) and subcloned into the expression vector pCDNA6/V5-HisB (Invitrogen).

(D) Generation of Stably Transfected Cell Lines.

MDCKII (strain II of Madin-Darby canine kidney) cells were cultured at 37° C. and 5% CO₂ in minimum essential medium Eagle supplemented with 10% fetal bovine serum, penicillin (100 U/ml), and streptomycin (100 μg/ml). As control cells MDCKII cells transfected with the empty vector (Cui et al., 1999) were used. For generation of the ABCC2/OATP2B1 double-transfected MDCKII cells the ABCC2 single-transfected MDCKII cells (Cui et al., 1999) were transfected with the expression vector pcDNA3.1/Hygro(−) containing the OATP2B1 cDNA by the metafectene method (Biontex, München, Germany). Selection was carried out with hygromycin (0.5 mg/ml). Cells of the clone showing the highest expression of ABCC2 and a homogeneous expression of OATP2B1 were designated ABCC2/OATP2B1 double-transfected MDCKII cells and chosen for further studies.

For generation of the quadruple-transfected MDCKII cells, first the ABCC2/OATP1B1 double-transfected MDCKII cells (Fehrenbach et al., 2003) were transfected with the OATP1B3 cDNA; selection was carried out with zeocin (1 mg/ml). Then, the clone stably expressing ABCC2, OATP1B1, and OATP1B3 was transfected with the expression vector pcDNA6/V5-HisB containing the cDNA encoding OATP2B1 and selection was carried out with blasticidin (10 μg/ml). Cells of the clone showing the highest expression of ABCC2 and, in addition, strong and widespread expression of OATP1B1, OATP1B3, and OATP2B1 were designated ABCC2-OATP1B1/1B3/2B1 quadruple-transfected MDCKII cells and used for further studies. Furthermore, cells of the clone showing the highest expression of MRP2 and, in addition, homogeneous expression of OATP1B1, OATP1B3 and OATP2B1 were designated as quadruple transfected cells and “MDCK-MRP2-OATP1B1-OATP1B3-OATP2B1 Quadruple Transfected Cells”, respectively, and used for further experiments.

(E) Cell Culture Conditions

For long term cultivation all MDCKII transfectants were cultured in minimum essential medium Eagle supplemented with 10% fetal bovine serum, penicillin, and streptomycin as described above. The vector-transfected MDCKII cells and the ABCC2 single-transfected MDCKII cells were grown in medium supplemented with G418 (1 mg/ml). The ABCC2/OATP1B1, ABCC2/OATP1B3, and ABCC2/OATP2B1 double-transfected MDCKII cells were grown in medium supplemented with G418 (1 mg/ml) and hygromycin (0.5 mg/ml). The ABCC2-OATP1B1/1B3/2B1 quadruple-transfected MDCKII cells were grown in medium which contains, in addition to G418 (1 mg/ml) and hygromycin (0.5 mg/ml), zeocin (0.1 mg/ml) and blasticidin (5 μg/ml). The selection agents were only used during long-term culture, but not after seeding of the cells on ThinCert filter membrane supports (see below).

(F) Immunoblot Analysis

Crude membrane fractions from transfected cells were prepared as described (Cui et al., 1999). The proteins were separated by SDS-polyacrylamide gel electrophoresis and blotted on nitrocellulose membranes (Amersham Biosciences). The antiserum EAG5 for detection of ABCC2 was used at a dilution of 1:5,000, the antisera ESL and SKT for detection of OATP1B1 and OATP1B3, respectively, were used at a dilution of 1:8,000. The antiserum SPA directed against OATP2B1 was used at a dilution of 1:40,000. Horseradish peroxidase-conjugated goat anti-rabbit IgG antiserum was used at a dilution of 1:3,000.

(G) Immunofluorescence Microscopy

(a) Human liver samples were snap-frozen in liquid nitrogen and stored at −80° C. until use. Serial cryosections of 6-μm thickness were cut and allowed to dry at room temperature for 30 minutes. After fixation in phosphate-buffered formaldehyde (10%, w/v) for 10 minutes, incubation with the primary and secondary antibodies was performed.

MDCKII cells were grown on ThinCerts (6-mm diameter, pore size 0.4 μm, pore density 1×10⁸ per cm² (Greiner Bio-One, Frickenhausen, Germany) as described (Letschert et al. 2005) for 3 days at confluence and induced with 10 mM sodium butyrate for 24 h (Cui et al., 1999). Fixation and post-treatment was performed as described (Cui et al., 2001). The ABCC2-specific antibody M₂III-6 was used at a dilution of 1:25, the antisera ESL and SKT were used at a dilution of 1:50. For the detection of OATP2B1, the antiserum SPA was used at a dilution of 1:200. Both the Cy3-conjugated goat anti-mouse and the Alexa Fluor 488-conjugated goat anti-rabbit IgG were used at a dilution of 1:300. Confocal laser scanning microscopy was performed using a LSM 510 Meta apparatus from Carl Zeiss (Jena, Germany).

(b) The apical localization of MRP2 and the basolateral localization of OATP1B1, OATP1B3 and OATP2B1 in the quadruple transfectants was demonstrated by use of confocal laser scanning microscopy. The quadruple transfectants were grown on Transwell membrane inserts (6.5 mm diameter, 0.4 μm pore size, Corning Costar, Bodenheim, Germany) for 3 days at confluency and subsequently induced with 10 mM sodium butyrate for 24 h (Cui et al., 1999). Cells were fixed with 2.5% paraformaldehyde in PBS (137 mM NaCl, 2.7 mM KCl, 8.0 mM Na₂HPO₄, 1.5 mM KH₂PO₄, pH 7.4) for 20 min as described in König et al., Am. J. Physiol. Gastrointest. Liver Physiol. 278 (2000), G156-64. Stable expression of the transporters in the quadruple transfectants was determined by use of polyclonal antisera ESL (König et al., Am. J. Physiol. Gastrointest. Liver Physiol. 278 (2000), G156-64) and SKT (König et al., J. Biol. Chem. 275 (2000), 23161-8) at a dilution of 1:50. MRP2 was determined in the quadruple transfected cells by use of the commercially available monoclonal antibody M₂III6 (Alexis). For detection of OATP2B1, a polyclonal antiserum (SPA) was prepared which was directed against the C-terminal amino acid sequence of the protein OATP2B1 (SPAVEQQLLVSGPGKKPEDSRV). For detection, the antiserum SPA was used at a dilution of 1:200.

(H) Transport Studies

(a) MDCKII cells were grown on ThinCerts (24-mm diameter, pore size 0.4 μm, pore density 1×10⁸ per cm²) and induced with butyrate as mentioned above. The cells were washed in prewarmed (37° C.) transport buffer (142 mM NaCl, 5 mM KCl, 1 mM KH₂PO₄, 1.2 mM MgSO₄, 1.5 mM CaCl₂, 5 mM glucose, and 12.5 mM HEPES, pH 7.3). The ³H-labeled substrate was dissolved in transport buffer and added either to the apical or to the basolateral compartment at the concentration indicated. After incubation at 37° C., the radioactivity in the opposite compartment was measured by sampling aliquots from the apical or basolateral compartment. Cells were washed 2 times with ice-cold transport buffer containing 0.5% bovine serum albumin and 3 times in ice-cold transport buffer. Intracellular accumulation of radioactivity was calculated by lyzing the cells with 0.2% sodium dodecyl sulfate (SDS) and measuring the radioactivity in the lysate.

The transcellular leakage was determined by addition of 1 μM [³H]inulin to the basolateral compartment and measurement of the radioactivity appearing in the apical compartment after an incubation period of 30 min. The transcellular leakage was 1-2% of the radioactivity added for all MDCKII cell clones examined in this study.

Transport of estrone 3-sulfate into inside-out membrane vesicles was performed using nick spin columns as described (Keppler et al., 1998). Membrane vesicles were prepared from MDCKII vector-transfected control cells and MDCKII-ABCC2 single transfected cells described by Cui et al. (1999) and Letschert et al. (2005). ATP-dependent transport was determined by subtracting values obtained in the presence of 5′-AMP from those in the presence of ATP.

(b) Transport assays were further carried out as described in Cui et al., Mol. Pharmacol. 60 (2001), 934-43. The apical localization of MRP2 and the basolateral expression of OATP1B1, OATP1B3 and OATP2B1 in the quadruple transfectants was shown by the generation of polar proliferation of the cells after initial growing on Transwell membrane inserts (24 mm diameter, 0.4 μM pore size, Corning Costar) and by use of confocal laser scanning microscopy. The transcellular transport of substrates on the Transwell membranes was demonstrated by use radioactively labelled bromosulfophthalein (BSP). BSP is a substrate for all of the transporters of the quadruple transfectant and is transported from the basolateral compartment into the apical compartment of the Transwell membrane inserts. In addition, part of the substance accumulates intracellularly.

### EXAMPLE 2

### Localization of Recombinantly Expressed Transporters MRP2, OATP1B1, OATP1B3 and OATP2B1 in MDCKII Cells

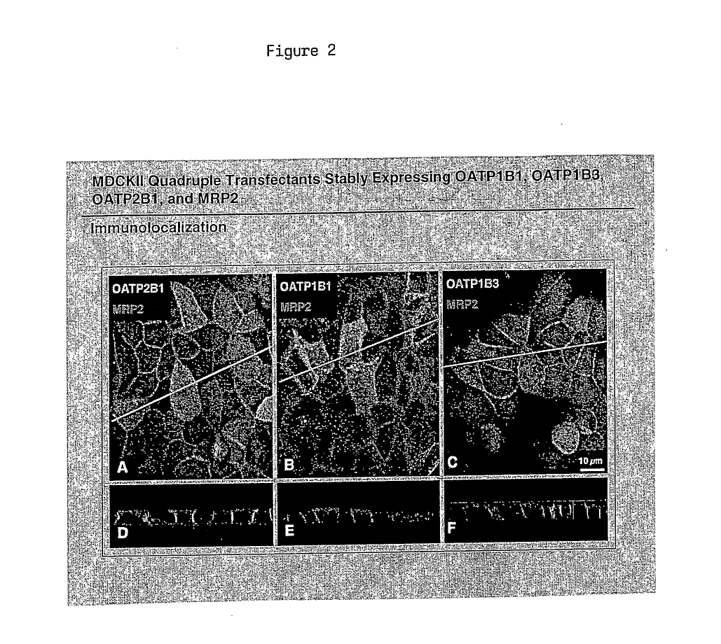

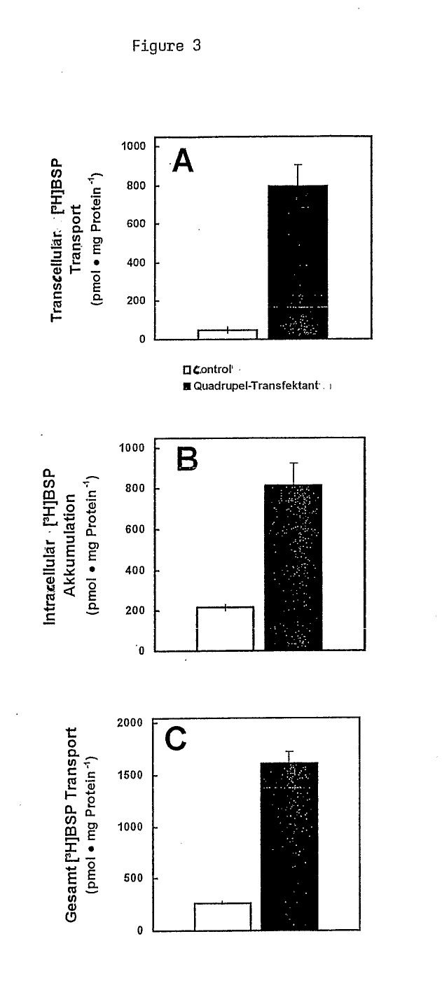

The immunolocalization of recombinantly expressed transporters MRP2, OATP1B1, OATP1B3 and OATP2B1 in quadruple transfected MDCKII cells was determined by confocal laser scanning microscopy in horizontal sections of the cells. Green fluorescence is indicative of OATP2B1 (FIG. 2A), OATP1B1 (FIG. 2B) and OATP1B3 (FIG. 2C), red fluorescence in FIGS. 2A-C is indicative of MRP2. FIGS. 2 D, E and F are vertical sections of the cells showing the basolateral localization of OATP1B1, OATP1B3 and OATP2B1 as well as the apical localization of MRP2 within the quadruple transfectants.

### EXAMPLE 3

### Transcellular Transport of [³H]BSP Mediated by OATP1B1, OATP1B3, OATP2B1 and MRP2

The function of human OATP1B1, OATP1B3, OATP2B1 and MRP2 in the quadruple-transfected cells was studied by measurement of the transcellular transport of the organic anion [³H]BSP. Thus, polarized MDCKII cells grown on Transwell membrane inserts were incubated with [³H]BSP at a concentration of 5 μM in the basolateral compartments. After 15 min., the radioactivity accumulated in the apical compartment and intracellularly was measured.

### EXAMPLE 4

### Expression and Localization of Recombinant ABCC2 and OATP Proteins in MDCKII Cells

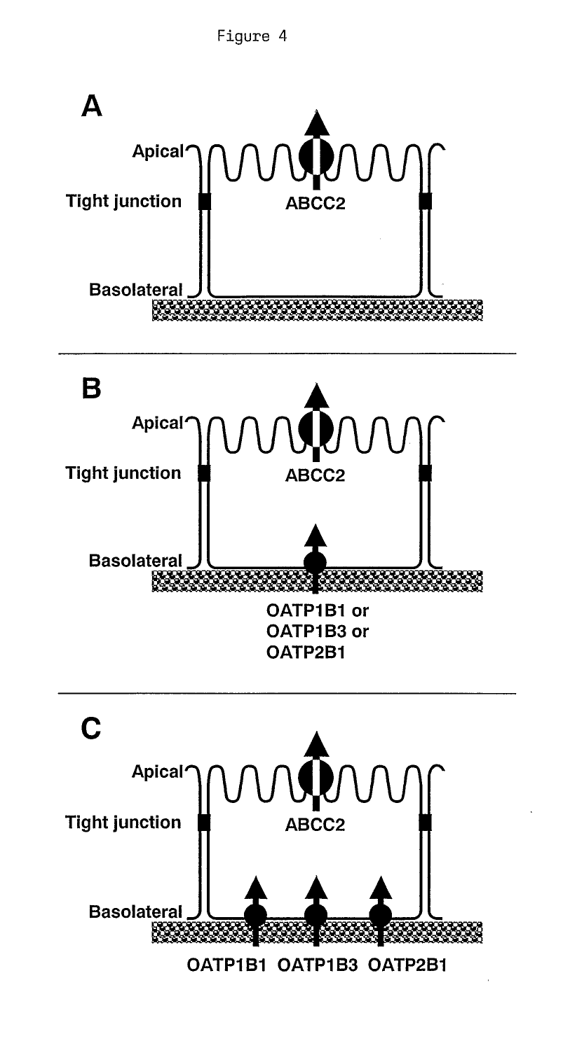

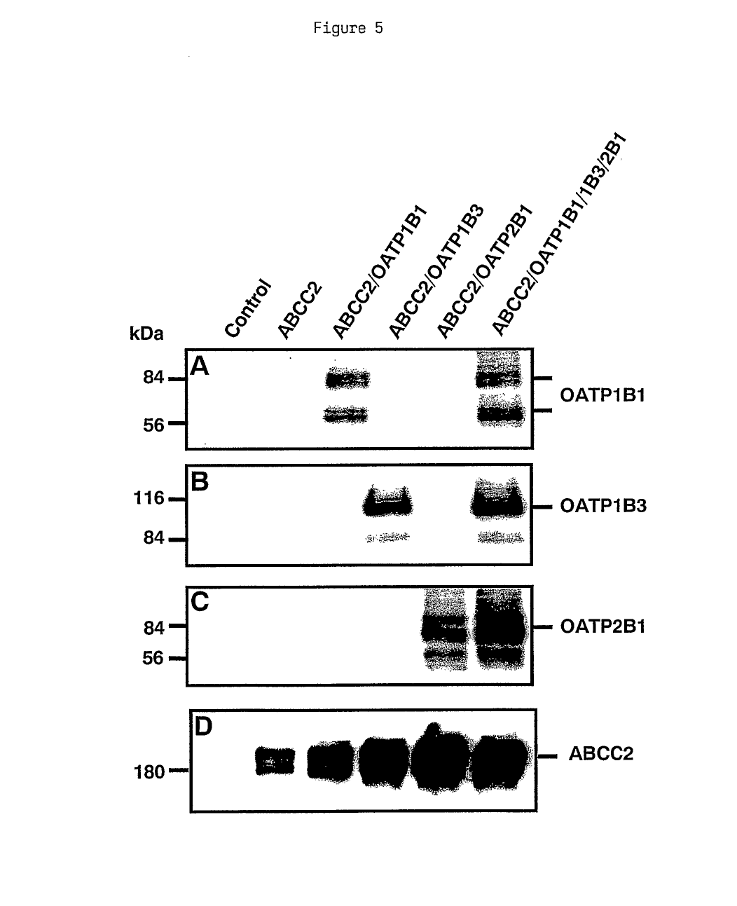

A scheme of the localization of the 4 transporters that were expressed in MDCKII cells is shown in FIG. 4. In the ABCC2/OATP1B1/1B3/2B1 quadruple-transfected MDCKII cells, the apical export pump ABCC2 and all 3 basolateral hepatic uptake transporters, OATP1B1, OATP1B3, and OATP2B1, were expressed (FIG. 4C). The levels of ABCC2 and the OATP proteins in the transfected MDCKII cells were analyzed by immunoblotting (FIG. 5). The OATP1B1 protein was detected by the antiserum ESL as two bands with apparent molecular masses of 56 kDa and 84 kDa in the ABCC2/OATP1B1 double-transfected MDCKII cells and in the ABCC2/OATP1B1/1B3/2B1 quadruple-transfected MDCKII cells. The OATP1B3 protein was present predominantly as fully glycosylated protein with an apparent molecular mass of 120 kDa in the ABCC2/OATP1B3 double-transfected MDCKII cells and in the ABCC2/OATP1B1/1B3/2B1 quadruple-transfected MDCKII cells (FIG. 5B). A minor portion of recombinant OATP1B3 was detected with an apparent molecular mass of about 84 kDa, which corresponds to its underglycosylated form (König et al., 2000b; Letschert et al., 2004). The OATP2B1 protein was detected with apparent molecular masses of about 84 kDa and 56 kDa in the ABCC2/OATP2B1 double-transfected MDCKII cells and in the ABCC2/OATP1B1/1B3/2B1 quadruple-transfected MDCKII cells (FIG. 5C). OATP2B1 appeared to be more strongly expressed in the ABCC2/OATP1B1/1B3/2B1 quadruple-transfected MDCKII cells than in the ABCC2/OATP2B1 double-transfected MDCKII cells. ABCC2 was strongly expressed in all transfectants except MDCKII vector-transfected control cells (FIG. 5D). In the vector-transfected control cells, none of the 4 transport proteins was detectable.

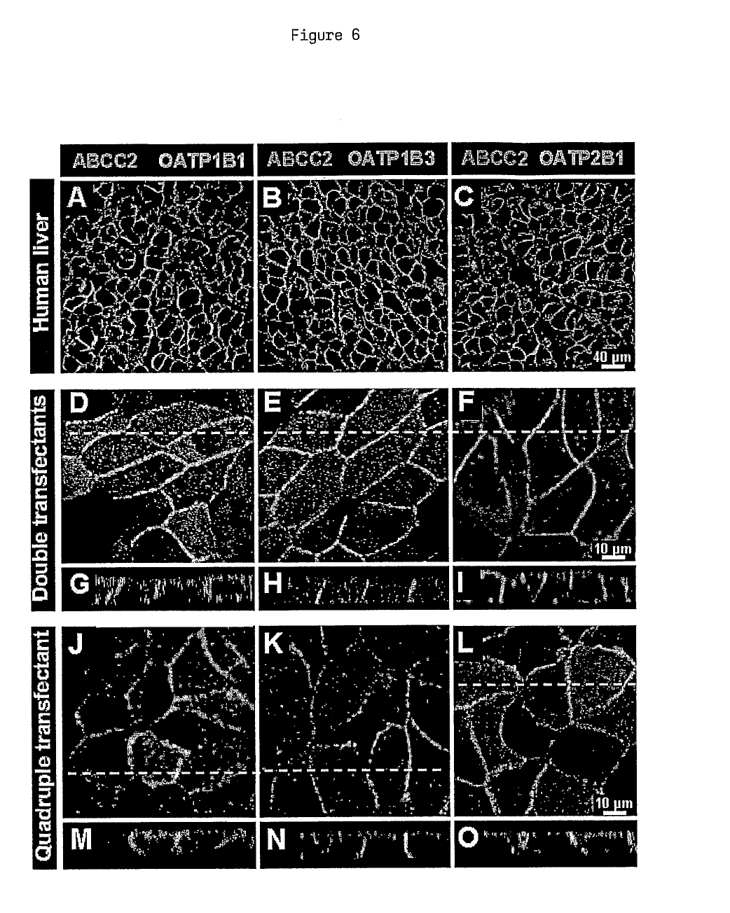

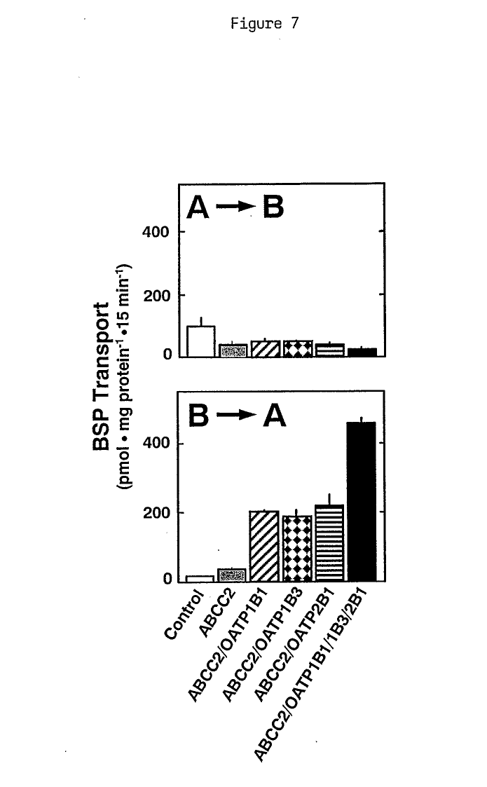

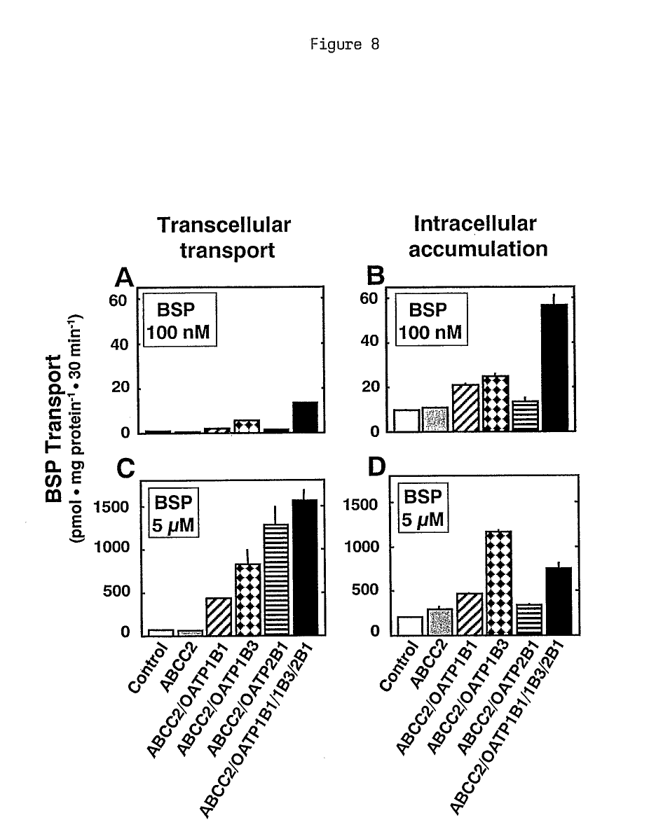

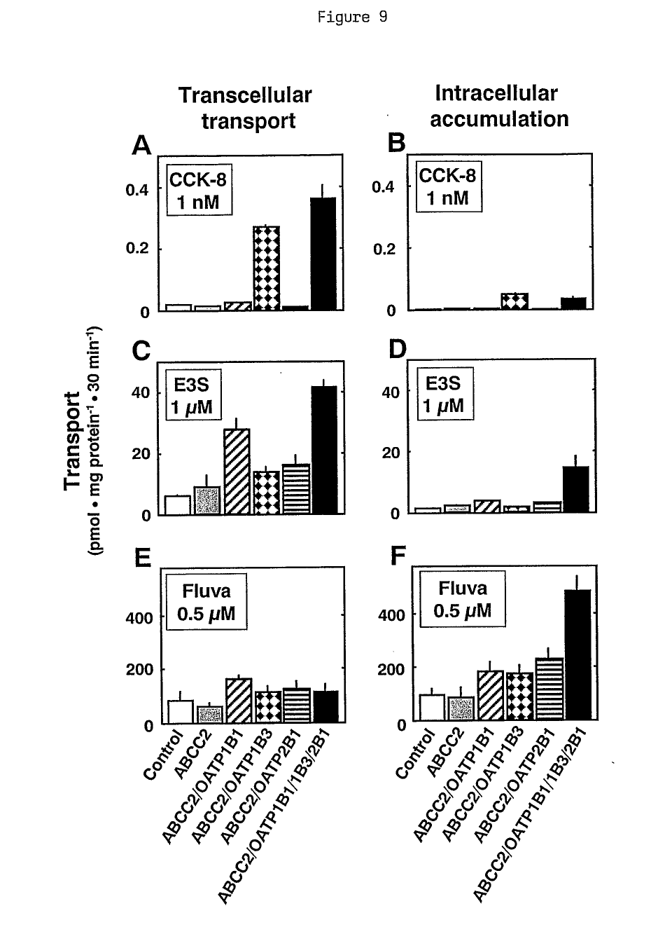

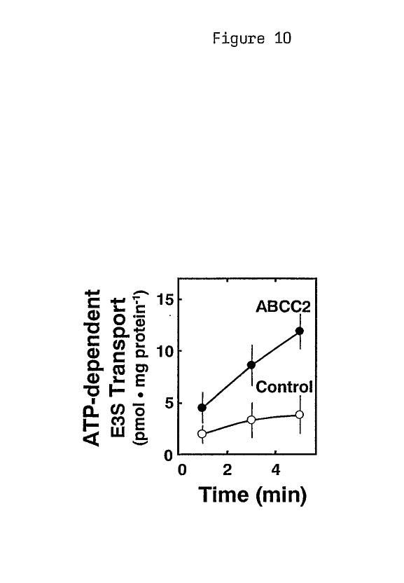

The cellular localization of the recombinant transporter proteins in the MDCKII transfectants and in human liver was studied by confocal laser scanning immunofluorescence microscopy (FIG. 6). Using the monoclonal antibody M₂III-6 directed against ABCC2 (red fluorescence), we performed a co-staining with the OATP proteins (green fluorescence) in the MDCKII transfectants and in human liver cryosections. In human liver, ABCC2 was localized to the bile canaliculi representing the apical domain of the hepatocyte membrane (FIG. 6A-C), whereas OATP1B1 (FIG. 6A), OATP1B3 (FIG. 6B), and OATP2B1 (FIG. 6C) were localized to the basolateral (sinusoidal) membrane domain of the hepatocytes.

ABCC2 was localized exclusively to the apical membrane domain of the ABCC2/OATP1B1, ABCC2/OATP1B3, and ABCC2/OATP2B1 double-transfected MDCKII cells, as well as of the ABCC2/OATP1B1/1B3/2B1 quadruple-transfected MDCKII cells, whereas the OATP proteins were localized to the basolateral membrane (FIG. 6D-O). The strict difference in domain-specific localization was particularly evident in the vertical sections of the cells by the lack of co-localization of ABCC2 with OATP1B1 (FIG. 6, G and M), or OATP1B3 (FIG. 6, H and N), or OATP2B1 (FIG. 6, I and O). The percentage of quadruple-transfected MDCKII cells staining positive for transporter proteins detected by confocal laser scanning immunofluorescence microscopy amounted to 45 % for ABCC2, to 82% for OATP1B1, to 96% for OATP1B3, and up to 100% for OATP2B1, based on, counting of at least 700 cells per clone.

### EXAMPLE 5

### Transcellular Transport of Organic Anions by ABCC2/OATP1B1/1B3/2B1 Quadruple-transfected MDCKII Cells

The transport properties were compared between the ABCC2/OATP1B1/1B3/2B1 quadruple-transfected MDCKII cells, double-transfected ABCC2/OATP1B1, or ABCC2/OATP1B3, or ABCC2/OATP2B1 MDCKII cells, single-transfected ABCC2 MDCKII cells, and vector-transfected control cells. [³H]BSP was used as model compound because it is an established substrate for ABCC2 (Cui et al., 2001), OATP1B1, OATP1B3 (König et al., 2000b), and OATP2B1 (Kullak-Ublick et al., 2001). The transcellular transport of [³H]BSP as a vectorial process in the MDCKII transfectants is shown in FIG. 7. The ABCC2 single-transfected MDCKII cells showed no significant transport when compared to the vector-transfected control cells. In the ABCC2/OATP1B1, ABCC2/OATP1B3, ABCC2/OATP2B1 double-transfected MDCKII cells, and in the ABCC2/OATP1B1/1B3/2B1 quadruple-transfected MDCKII cells, a basolateral to apical transport of [³H]BSP was observed, whereas the respective apical to basolateral transport was negligible. We compared the transcellular transport rates and intracellular accumulation of [³H]BSP at two substrate concentrations (FIG. 8). At 100 nM [³H]BSP, an intracellular [³H]BSP accumulation was observed and only minimal transcellular transport was detected in the double-transfected MDCKII cells (FIG. 8, A and B). The transcellular transport as well as the intracellular accumulation was highest in the ABCC2/OATP1B1/1B3/2B1 quadruple-transfected MDCKII cells. Using [³H]BSP at a concentration of 5 μM, that is in the range of the Kₘ of OATP2B1 for [³H]BSP (Kullak-Ublick et al., 2001) and ABCC2 (Cui et al., 2001), all 3 double-transfected MDCKII cell lines showed transcellular [³H]BSP transport, while the highest transcellular [³H]BSP transport was seen with the ABCC2/OATP1B1/1B3/2B1 quadruple-transfected MDCKII cells (FIG. 8, C and D). [³H]CCK-8 was tested as a substrate since it is considered a selective substrate of OATP1B3 (Ismair et al., 2001) and a substrate of ABCC2 (Letschert et al., 2005). [³H]CCK-8 was efficiently transported transcellularly by the ABCC2/OATP1B3 double-transfected MDCKII cells and by the ABCC2/OATP1B1/1B3/2B1 quadruple-transfected MDCKII cells (FIG. 9). Only a minor portion of the [³H]CCK-8 taken up by the cells, was retained intracellularly, due to the efficient apical export via ABCC2.

Vectorial transport of [³H]estrone 3-sulfate was detected in all 3 double-transfected MDCKII cells and in ABCC2/OATP1B1/1B3/2B1 quadruple-transfected MDCKII cells (FIG. 9, C and D) suggesting that [³H]estrone 3-sulfate is not only a substrate for OATP1B1, OATP1B3, and OATP2B1, but also for ABCC2. The transport of [³H]estrone 3-sulfate by ABCC2 was confirmed by transport studies using membrane vesicles prepared from MDCKII-ABCC2 single transfectants (FIG. 10). The ABCC2-mediated [³H]estrone 3-sulfate transport in the presence of ATP was 3.1-fold enhanced compared to controls.

[³H]Fluvastatin was accumulated intracellularly in the double-transfected cells and in ABCC2/OATP1B1/1B3/2B1 quadruple-transfected MDCKII cells (FIG. 9F). However, no significant vectorial transport of [³H]fluvastatin was observed in the transfectants when compared to the control cells. (FIG. 9E).

The kinetic constants for the intracellular accumulation of [³H]BSP and [³H]fluvastatin are summarized in Table 1. [³H]Fluvastatin was transported with high affinity by all 3 OATPs. OATP2B1, with a Kₘ value of 0.7±0.3 μM showed the highest affinity for [³H]fluvastatin relative to OATP1B1 (Kₘ 2.4±0.9 μM) and OATP1B3 (Kₘ 7.0±2.5 μM). Co-expression of all 3 OATP transporters in the quadruple-transfected MDCKII cells resulted in an enhanced transport capacity indicated by the Vₘₐₓ values, but the substrate affinity was in the range of the separate OATPs (Table 1). The Kₘ value of 4.8±0.2 μM for [³H]BSP determined in the ABCC2/OATP1B1/1B3/2B1 quadruple-transfected MDCKII cells was also in the same range as the Kₘ determined in the respective double-transfected MDCKII cells (Table 1).

TABLE 1

| | Bromosulfophthalein | | Fluvastatin | |
|---|---|---|---|---|
| | | Vₘₐₓ | | Vₘₐₓ |
| | | pmol · mg | | pmol · mg |
| | Kₘ | protein⁻¹ · | Kₘ | protein⁻¹ · |
| Cell line | μM | min⁻¹ | μM | min⁻¹ |
| ABCC2-OATP1B1 | 2.4 ± 0.7 | 41.5 ± 5.2 | 2.4 ± 0.9 | 43.4 ± 6.7 |
| ABCC2-OATP1B3 | 2.2 ± 0.3 | 82.7 ± 4.0 | 7.0 ± 2.5 | 53.6 ± 11.0 |
| ABCC2-OATP2B1 | 3.4 ± 1.2 | 52.8 ± 8.6 | 0.7 ± 0.3 | 23.0 ± 2.6 |
| ABCC2-OATP1B1/ | 4.8 ± 0.2 | 197.8 ± 3.0 | 2.3 ± 0.4 | 93.9 ± 7.4 |
| 1B3/2B1 | | | | |

### REFERENCES

Abe T, Kakyo M, Tokui T, Nakagomi R, Nishio T, Nakai D, Nomura H, Unno M, Suzuki M, Naitoh T, Matsuno S, Yawo H (1999) Identification of a novel gene family encoding human liver-specific organic anion transporter LST-1. J Biol Chem 274:17159-17163.

Büchler M, König J, Brom M, Kartenbeck J, Spring H, Horie T, Keppler D (1996) cDNA cloning of the hepatocyte canalicular isoform of the multidrug resistance protein, cMrp, reveals a novel conjugate export pump deficient in hyperbilirubinemic mutant rats. J Biol Chem 271:15091-15098.

Cui Y, König J, Buchholz J K, Spring H, Leier I, Keppler D (1999) Drug resistance and ATP-dependent conjugate transport mediated by the apical multidrug resistance protein, MRP2, permanently expressed in human and canine cells. Mol Pharmacol 55:929-937.

Cui Y, König J, Keppler D (2001) Vectorial transport by double-transfected cells expressing the human uptake transporter SLC21A8 and the apical export pump ABCC2. Mol Pharmacol 60:934-943.

Evers R, Kool M, van Deemter L, Janssen H, Calafat J, Oomen L C, Paulusma C C, Oude Elferink R P, Baas F, Schinkel A H, Borst P (1998) Drug export activity of the human canalicular multispecific organic anion transporter in polarized kidney MDCK cells expressing cMOAT (MRP2) cDNA. J Clin Invest 101:1310-1319.

Fehrenbach T, Cui Y, Faulstich H, Keppler D (2003) Characterization of the transport of the bicyclic peptide phalloidin by human hepatic transport proteins. Naunyn Schmiedebergs Arch Pharmacol 368:415-420.

Hagenbuch B, Meier P J (2004) Organic anion transporting polypeptides of the OATP/SLC21 family: phylogenetic classification as OATP/SLCO superfamily, new nomenclature and molecular/functional properties. Pflugers Arch 447:653-665

Hsiang B, Zhu Y, Wang Z, Wu Y, Sasseville V, Yang W P, Kirchgessner T G (1999) A novel human hepatic organic anion transporting polypeptide (OATP2). Identification of a liver-specific human organic anion transporting polypeptide and identification of rat and human hydroxymethylglutaryl-CoA reductase inhibitor transporters. J Biol Chem 274:37161-37168.

Ismair M G, Stieger B, Cattori V, Hagenbuch B, Fried M, Meier P J, Kullak-Ublick G A (2001) Hepatic uptake of cholecystokinin octapeptide by organic anion-transporting polypeptides OATP4 and OATP8 of rat and human liver. Gastroenterology 121:1185-1190.

Keppler D, Kartenbeck J (1996) The canalicular conjugate export pump encoded by the cmrp/cmoat gene. Prog Liver Dis 14:55-67.

Keppler D, Jedlitschky G, Leier I (1998) Transport function and substrate specificity of multidrug resistance protein. Methods Enzymol 292:607-616.

König J, Nies A T, Cui Y, Leier I, Keppler D (1999) Conjugate export pumps of the multidrug resistance protein (MRP) family: localization, substrate specificity, and MRP2-mediated drug resistance. Biochim Biophys Acta 1461:377-394.

König J, Cui Y, Nies A T, Keppler D (2000a) A novel human organic anion transporting polypeptide localized to the basolateral hepatocyte membrane. Am J Physiol Gastrointest Liver Physiol 278:G156-164.

König J, Cui Y, Nies A T, Keppler D (2000b) Localization and genomic organization of a new hepatocellular organic anion transporting polypeptide. J Biol Chem 275:23161-23168.

Kullak-Ublick G A, Ismair M G, Stieger B, Landmann L, Huber R, Pizzagalli F, Fattinger K, Meier P J, Hagenbuch B (2001) Organic anion-transporting polypeptide B (OATP-B) and its functional comparison with three other OATPs of human liver. Gastroenterology 120:525-533.

Letschert K, Keppler D, König J (2004) Mutations in the SLCO1B3 gene affecting the substrate specificity of the hepatocellular uptake transporter OATP1B3 (OATP8). Pharmacogenetics 14:441-452.

Letschert K, Komatsu M, Hummel-Eisenbeiss J, Keppler D (2005) Vectorial transport of the peptide CCK-8 by double-transfected MDCKII cells stably expressing the organic anion transporter OATP1B3 (OATP8) and the export pump ABCC2 (MRP2). J Pharmacol Exp Ther 313:549-556.

Sasaki M, Suzuki H, Ito K, Abe T, Sugiyama Y (2002) Transcellular transport of organic anions across a double-transfected Madin-Darby canine kidney II cell monolayer expressing both human organic anion-transporting polypeptide (OATP2/SLC21A6) and Multidrug resistance-associated protein 2 (MRP2/ABCC2). J Biol Chem 277:6497-6503.

Sasaki M, Suzuki H, Aoki J, Ito K, Meier P J, Sugiyama Y (2004) Prediction of in vivo biliary clearance from the in vitro transcellular transport of organic anions across a double-transfected Madin-Darby canine kidney II monolayer expressing both rat organic anion transporting polypeptide 4 and multidrug resistance associated protein 2. Mol Pharmacol 66:450-459.

Schaub T P, Kartenbeck J, König J, Spring H, Dorsam J, Staehler G, Störkel S, Thon W F, Keppler D (1999) Expression of the MRP2 gene-encoded conjugate export pump in human kidney proximal tubules and in renal cell carcinoma. J Am Soc Nephrol 10:1159-1169.

Spears K J, Ross J, Stenhouse A, Ward C J, Goh L B, Wolf C R, Morgan P, Ayrton A, Friedberg T H (2005) Directional trans-epithelial transport of organic anions in porcine LLC-PK1 cells that co-express human OATP1B1 (OATP-C) and MRP2. Biochem Pharmacol 69:415-423.

Suzuki H, Sugiyama Y (2000) Transport of drugs across the hepatic sinusoidal membrane: sinusoidal drug influx and efflux in the liver. Semin Liver Dis 20:251-263.

Tamai I, Nezu J, Uchino H, Sai Y, Oku A, Shimane M, Tsuji A (2000) Molecular identification and characterization of novel members of the human organic anion transporter (OATP) family. Biochem Biophys Res Commun 273:251-260.

Tse F L, Dain J G, Kalafsky G (1995) Disposition of [³H]fluvastatin following single oral doses in beagle dogs and rhesus monkeys with bile fistulae. Biopharm Drug Dispos. 16:211-219.

1. A cell line comprising (a) three DNA sequences encoding different uptake transporters for organic anions operatively linked with a promoter and (b) a DNA sequence encoding an export pump for organic anions or anionic conjugates operatively linked with a promoter.

2. The cell line of claim 1 comprising at least one further heterologous DNA sequence.

3. The cell line of claim 1 which is a canine or human cell line and the DNA sequences of (a) and/or (b) are human.

4. The cell line of claim 1 which is a kidney cell line.

5. The cell line of claim 1 wherein the uptake transporters for organic anions are members of the subgroup SLCO (21A) or 22A of the solute carrier (SLC) superfamily.

6. The cell line of claim 5, wherein the uptake transporters for organic anions are OATP1B1, OATP1B3 and OATP2B1.

7. The cell line of claim 1 wherein the export pump for organic anions or anionic conjugates is a member of the MDR (ABCB) subgroup or the MRP (ABCC) subgroup of the ABC superfamily.

8. The cell line of claim 7, wherein the export pump for organic anions or anionic conjugates is the bile salt export pump BSEP (ABCB1I) or the multidrug resistance protein 2 (MRP2; ABCC2).

9. The cell line of claim 1 wherein the DNA sequences encoding uptake transporters for organic anions and/or the DNA sequence encoding an export pump for organic anions or anionic conjugates are operatively linked with a promoter allowing high expression.

10. The cell line of claim 1, wherein it is a quadruple transfected cell line.

11. A method for for the identification of a transport substrate or a transport inhibitor, the method comprising:
(a) providing a cell line comprising:
(i) three DNA sequences encoding different uptake transporters for organic anions operatively linked with a promoter; and
(ii) a DNA sequence encoding an export pump for organic anions or anionic conjugates operatively linked with a promoter,
(b) introducing a compound into the cell line;
(c) determining if the compound is a transport substrate or transport inhibitor.

12. The method according to claim 11 wherein the transport inhibitor is a drug candidate.

13. The method according to claim 11 wherein the identification of a transport substrate or a transport inhibitor is carried out as high throughput screening.

14. The method according to claim 11 wherein the uptake transporters for organic anions are OATP1B1, OATP 1B3 and OATP2B1.

15. The method according to claim 14, wherein the export pump for organic anions or anionic conjugates is a member of the MDR (ABCB) subgroup or the MRP (ABCC) subgroup of the ABC superfamily.

16. The cell line of claim 6 wherein the export pump for organic anions or anionic conjugates is a member of the MDR (ABCB) subgroup or the MRP (ABCC) subgroup of the ABC superfamily.

17. The cell line of claim 16 wherein the DNA sequences encoding uptake transporters for organic anions and/or the DNA sequence encoding an export pump for organic anions or anionic conjugates are operatively linked with a promoter allowing high expression.

18. The cell line of claim 7 wherein the uptake transporters for organic anions are members of the subgroup SLCO (21A) or 22A of the solute carrier (SLC) superfamily.

19. A method for making a cell line the identification of a transport substrate or a transport inhibitor, the method comprising:
(a) providing a vector comprising:
(i) three DNA sequences encoding different uptake transporters for organic anions operatively linked with a promoter; and
(ii) a DNA sequence encoding an export pump for organic anions or anionic conjugates operatively linked with a promoter;
(b) transfecting a host cell line to generate a transformed host cell; and
(c) maintaining the transformed host cell under biological conditions sufficient for expression of the DNA sequences.

20. The method of claim 19, wherein the uptake transporters for organic anions are OATP1B1, OATP1B3 and OATP2B1 and the export pump for organic anions or anionic conjugates is a member of the MDR (ABCB) subgroup or the MRP (ABCC) subgroup of the ABC superfamily.
